(19) United States
(12) Patent Application Publication
(10) Pub. No.: US 2026/0364372 A1
(43) Pub. Date: Nov. 19, 2026

(54) COST-BALANCED EIGHT-PULSE PHOTONIC THERAPY FOR RAPID MODULATION OF PROTEIN-FOLDING AND INFLAMMATORY PATHWAYS

(71) Applicant: Jonathan Washburn, Austin, TX (US)

(72) Inventor: Jonathan Washburn, Austin, TX (US)

(21) Appl. No.: 19/676,220

(22) Filed: May 13, 2026

Related U.S. Application Data
(60) Provisional application No. 63/805,874, filed on May 14, 2025.

(51) Int. Cl. A61N 5/06; A61B 17/00

(52) U.S. Cl. CPC A61N 5/0616; A61B 2017/00185; A61N 2005/0628; A61N 2005/0629; A61N 2005/0645; A61N 2005/0652

(57) ABSTRACT

Methods, devices, systems, and computer-readable media are disclosed for non-thermal photonic stimulation of biological tissue using an exact eight-sub-pulse burst architecture. Each treatment cycle comprises exactly eight optical sub-pulses arranged in mirror pairs across a cycle midpoint. Pulse-pair amplitude ratios are evaluated using a dimensionless J-cost symmetry residual, and an independent signed eight-slot ledger schedule is verified for equal-and-opposite neutrality across the cycle. Continued optical emission is permitted only while the symmetry residual satisfies a predetermined dimensionless threshold and the signed schedule satisfies a zero-sum neutrality condition. Preferred embodiments employ visible solid-state emitters, including GaN micro-LED implementations, direct optical feedback, rollback circuitry, and control logic configured to interrupt emission upon non-compliance. The disclosed architecture provides a cost-balanced, ledger-neutral, power-capped, and non-thermal photonic therapy framework with real-time verification and fault handling.

### CROSS-REFERENCE TO RELATED APPLICATIONS

This application claims the benefit of U.S. Provisional Patent Application No. 63/805,874, filed May 14, 2025, the entire disclosure of which is incorporated herein by reference.

### BACKGROUND OF THE INVENTION

The invention relates to photonic therapy devices, systems, firmware-controlled emitters, and treatment methods for non-thermal photobiomodulation of biological tissue, and more particularly to systems and methods that deliver mirror-symmetric eight-sub-pulse optical bursts whose pairwise amplitude ratios are audited by a J-cost functional and whose signed cycle schedule is verified for equal-and-opposite ledger neutrality before and during delivery to tissue. Low-level light therapy and photobiomodulation are known, but existing systems typically use empirically chosen continuous-wave or pulsed-light protocols without a formally constrained pulse architecture, without cycle-by-cycle symmetry verification, without explicit energetic or ledger accounting, and without machine-verified correctness conditions. Conventional systems may employ selected wavelengths, selected pulse rates, or selected duty cycles, but they generally do not provide the specific combination of an exact eight-sub-pulse mirror architecture, a dimensionless symmetry audit, an independent signed ledger-neutrality condition, and real-time enforcement logic that together govern whether emission is permitted to continue.

Existing protocols also suffer from arbitrary dosimetry. In many cases, power levels, pulse patterns, and treatment timing are chosen empirically rather than through a principled cost-accounting framework that constrains the internal balance of the delivered optical sequence. Existing systems further suffer from thermal confounds because some treatment regimes introduce enough heating that thermal and photochemical effects are difficult to separate, thereby complicating both safety analysis and mechanism attribution.

Conventional phototherapy systems also lack cost accounting for the internal pulse structure itself. Prior systems do not audit the symmetry or balance of a pulse sequence using a formal residual derived from pulse-pair relationships, and they do not independently enforce a signed cycle schedule that guarantees equal-and-opposite balancing across the cycle. As a result, prior systems may monitor output generally, but they do not verify whether a delivered burst satisfies the particular architecture-level balance conditions disclosed herein.

Conventional systems further exhibit weak temporal structure. Prior pulsed-light systems do not exploit an exact eight-part cycle structure with mirror pairing and associated phase relationships as a load-bearing architectural requirement. They also do not employ the disclosed combination of exact cycle structure, pairwise mirror organization, dimensionless audit logic, and independent signed balancing as an integrated operating framework for tissue treatment.

Conventional systems additionally lack formal verification of protocol correctness. Prior phototherapy approaches do not provide machine-verified proof that the claimed pulse-balance conditions, threshold conditions, timing conditions, and related architectural constraints are actually satisfied by the disclosed treatment framework. Accordingly, a need remains for photonic therapy architectures that are non-thermal, power-capped, and governed by an exact audited pulse structure with real-time compliance enforcement rather than by generic pulse delivery and post hoc monitoring.

### SUMMARY OF THE INVENTION

The invention provides methods, devices, systems, kits, and computer-readable media for non-thermal photonic stimulation of biological tissue using a non-arbitrary, exact eight-sub-pulse burst architecture in which pulse pairs are constrained by exact mirror symmetry, pulse-pair amplitude ratios are audited by a dimensionless J-cost symmetry residual, an independent signed eight-slot schedule is verified for equal-and-opposite ledger neutrality, and continued emission is permitted only while the dimensionless symmetry residual satisfies a predetermined dimensionless threshold, the signed eight-slot schedule satisfies a zero-sum neutrality condition, and photon-budget, temperature, calibration, hardware, and watchdog safety conditions required for continued emission remain satisfied. The invention is therefore not generic pulsed phototherapy with monitoring. The invention is the exact, audited, cost-balanced, ledger-neutral eight-sub-pulse delivery architecture and the hardware and firmware that enforce it in real time. In the disclosed architecture, each treatment cycle contains exactly eight sub-pulses. The sub-pulses are indexed n=1 through 8 and are organized into mirror pairs (1,8), (2,7), (3,6), and (4,5). For the disclosed mirror architecture, the pulse intensities satisfy I_n=I_9-n for the mirror pairs, and the pulse phases satisfy phi_n=phi_9-n +pi for the mirror pairs. The dimensionless symmetry residual is defined as R_sym=J(I_1/I_8)+J(I_2/I_7)+J(I_3/I_6)+J(I_4/I_5), where J(x)=½(x+1/x)−1. The disclosed J-cost functional is the unique cost functional used in the architecture disclosed herein. Because J(x)>=0 for x>0 and J(x)=0 iff x=1, exact mirror equality yields exact zero residual.

Independently of the J-audit, the cycle carries a signed eight-slot schedule satisfying delta_0=−delta_4, delta_1=−delta_5, delta_2=−delta_6, and delta_3=−delta_7, such that the sum from k=0 to 7 of delta_k=0. The J-audit and the signed ledger schedule are independent, distinct, and complementary guarantees. The J-audit governs amplitude-ratio precision through a dimensionless residual. The signed ledger schedule governs equal-and-opposite signed balancing across the cycle. The invention uses both conditions in parallel, and a cycle can fail overall protocol compliance if either condition fails.

The invention further includes control logic that generates or retrieves the exact eight-sub-pulse waveform, applies the mirror architecture, computes the pulse-pair ratios, computes the dimensionless symmetry residual, verifies the signed schedule, evaluates photon-budget, temperature, calibration, hardware, and watchdog safety conditions, and either permits continued emission or places the system into one or more control outcomes including pause, inhibition, temporary shutdown, latched shutdown, termination, or rollback when any required condition is not satisfied. In preferred embodiments, audit compliance can be determined from commanded pulse values, measured pulse values, or both. When commanded values and measured values differ, compliance is determined from measured values for acceptance and fault handling, while commanded values can be retained for comparison, diagnostics, and calibration analysis.

In preferred embodiments, pause, inhibition, temporary shutdown, latched shutdown, termination, or rollback can be triggered by any one or more of mirror-symmetry failure, symmetry-residual threshold failure, signed-schedule failure, photon-budget exceedance, temperature-limit exceedance, calibration failure, hardware fault, or watchdog fault. The invention is therefore a dual-guarantee architecture with real-time enforcement and fault-handling logic rather than a generic balanced pulse system.

Preferred embodiments employ a visible GaN micro-LED carrier around 450+/−20 nm, a master timing reference around 125 Hz, a sub-burst rate around 1000 Hz, a treatment session of about 200 cycles, a per-sub-pulse photon cap of at most 1.5×10\hat{ }15 photons, a per-cycle photon cap of at most 1.2×10\hat{ }16 photons, a per-treatment photon cap of at most 2.4×10\hat{ }18 photons, and a tissue-interface temperature rise of less than about 0.3 C. These are preferred and strategically valuable fallback positions, not the universal definition of the invention.

In a broad fallback position, the mirror-pair amplitudes can be required to satisfy a predetermined mismatch tolerance and the dimensionless symmetry residual can be required to satisfy a predetermined threshold. In a narrower preferred fallback position, the mirror mismatch is within +/−0.5 percent and R_sym<=1/20000. In a narrower exact preferred implementation, the single-pair penalty at the +/−0.5 percent mismatch boundary is 1/80400 and the four-pair aggregate residual is 1/20100, which is strictly less than 1/20000. These exact values are exact, proof-backed support anchors for the preferred threshold regime and show that the preferred operating point is not arbitrary. In a broad fallback position, a treatment session can comprise one or more cycles. In a narrower fallback position, a treatment session can comprise a plurality of cycles. In a narrower preferred position, the treatment session comprises about 200 cycles. The exact values 1/80400, 1/20100, and 1/20000 are recited herein as exemplary, proof-backed support anchors for one preferred threshold regime and are not limiting; in alternative embodiments, R_tol can be selected to satisfy R_tol greater than 4*J(1+epsilon/100) for any predetermined mirror-pair mismatch tolerance epsilon, and the four-pair aggregate residual at the corresponding mismatch boundary remains strictly below R_tol by construction.

In a preferred embodiment, the carrier is about 450+/−20 nm and the corresponding photon energy is within a preferred tolerance band of the 31st multiple of E_coh. In a broad fallback position, the carrier can be selected relative to the disclosed coherence-quantum criterion. In a narrower fallback position, the carrier can be selected to satisfy a predetermined harmonic-proximity criterion relative to E_coh, a predetermined energy-band criterion relative to E_coh, or both. In a further narrower fallback position, the carrier is a visible carrier within the disclosed water-transparency window and is selected relative to the disclosed coherence-quantum criterion. In a preferred narrower position, the carrier is around 450+/−20 nm and is within the disclosed preferred tolerance band of the 31st multiple of E_coh. The disclosed coherence quantum is E_coh=phi\hat{ }−5. The exponent 5 is disclosed as forced by the stated conditions of the framework used herein. This forced exponent matters because it anchors the disclosed carrier-selection logic and supports the preferred harmonic-matching rationale. The preferred 450+/−20 nm carrier is therefore a proof-backed, non-arbitrary, and architecture-defining preferred implementation of the disclosed carrier-selection rule, not an arbitrary wavelength choice.

The specification includes formal support for J-cost non-negativity and zero condition, exact zero residual under exact mirror equality, exact residual values at the preferred tolerance boundary, signed schedule balance, the forced exponent in E_coh=phi\hat{ }−5, 450 nm harmonic matching to E_coh, substrate overlap with water and protein H-bond bands, timing values, photon-count values, and a combined machine-verified viability certificate. These proof-backed items are technical support for the architecture, for the preferred thresholds, and for the non-arbitrary selection of the preferred implementation package. These proof-backed items are not substitutes for claim language, but they should appear in the specification as architectural credibility anchors and should not be isolated only in a later proof section.

Downstream embodiments can include wound-healing bandages, clinical probes, tabletop skin-treatment units, wearable vascular patches, kit systems, calibration articles, telemetry software, and cloud audit platforms. The wound-healing bandage and joint probe can be described as preferred downstream embodiments. The tabletop skin-treatment unit and wearable vascular patch can be described as downstream embodiments that remain subordinate to the core exact audited eight-sub-pulse architecture. Cloud audit platforms, telemetry software, and related infrastructure can be described tersely as optional adjunct systems rather than as a separate inventive center.

### BRIEF DESCRIPTION OF THE DRAWINGS

FIG. 1 depicts a mirror-symmetric eight-sub-pulse burst architecture showing a treatment cycle containing exactly eight sub-pulses arranged symmetrically about a midpoint, together with mirror-pair groupings and labels indicating the disclosed intensity and phase relations.

FIG. 2 depicts a J-cost audit computation flow in which pulse measurement inputs are processed through pair-ratio calculators, J evaluators, a residual summation block, and a threshold comparator to generate a pass or fail audit output.

FIG. 3 depicts a signed eight-slot ledger schedule in which eight signed slots are arranged in equal-and-opposite anti-mirror relations such that the schedule produces a zero-sum output.

FIG. 4 depicts an overall system block diagram for an audited photonic therapy device including a clock module, controller, emitter array, verifier, photon feedback subsystem, rollback gate, coupling interface, target tissue, telemetry output, and power subsystem.

FIG. 5 depicts a timing architecture and treatment session sequence including a master clock, cycle period, sub-pulse sequence, session start, session end, preferred cycle count, active emission interval, and optional repeat interval.

FIG. 6 depicts a preferred hardware embodiment including a phi-clock oscillator, GaN micro-LED array, FPGA verifier, microcontroller, PIN-diode set, rollback circuitry, hydrogel pad, enclosure, and patient-contact side.

FIG. 7 depicts a firmware and control workflow progressing through session initiation, waveform generation, symmetry check, J-audit, ledger check, dose and thermal check, continue decision, rollback decision, logging, and session completion.

FIG. 8 depicts carrier-energy and substrate-support relationships showing an E_coh marker, a carrier-energy marker, a harmonic relation indicator, a water H-bond band, a protein H-bond band, a water transparency window, and a substrate-support output.

FIG. 9 depicts a machine-verification and proof dependency architecture in which J-cost support, eight-tick support, coherence exponent support, substrate-support, ledger-neutrality support, and timing and dose support feed a master certificate for the disclosed viability package.

FIG. 10 depicts worked numerical examples and audit thresholds including a harmonic-matching panel, a residual-threshold panel, a photon-budget panel, a timing panel, and a thermal-budget panel.

FIG. 11 depicts a demonstrated pilot-results summary including a wound-closure panel, a cytokine panel, a thermal-observation panel, and a preliminary-data notice identifying the data as empirical and not machine-verified.

FIG. 12 depicts a wound-healing bandage embodiment including a bandage body, emitter region, hydrogel pad, power source, controller package, status indicator, and wound site.

FIG. 13 depicts a joint-inflammation probe embodiment including a probe housing, treatment tip, emitter array, contact sensor, display, battery, and data or charging port.

FIG. 14 depicts a tabletop collagen-treatment embodiment including a base unit, articulated arm, treatment head, enlarged emitter array, thermal monitor, and control console.

FIG. 15 depicts a wearable vascular patch embodiment including a flexible patch body, emitter array, adhesive layer, vascular target region, optional PPG sensor, and controller package.

### DETAILED DESCRIPTION OF THE INVENTION

The present disclosure describes systems, devices, methods, kits, and control architectures for non-thermal photonic stimulation of biological tissue using an exact, audited, cost-balanced, ledger-neutral eight-sub-pulse delivery architecture. In the disclosed architecture, each treatment cycle is implemented as an exact eight-sub-pulse burst 100, and continued emission is permitted only while the cycle remains compliant with both a dimensionless symmetry audit and an independent signed ledger-neutrality condition. The invention is therefore centered on the exact audited burst architecture itself, rather than on any particular indication, commercial packaging format, or downstream deployment scenario. The same exact audited architecture can be embodied in patch-type devices, handheld devices, stationary devices, kits, and software-controlled treatment hardware while preserving the same disclosed cycle structure, verification logic, control hierarchy, and enforcement behavior. The resulting technical improvement is a real-time emission interlock and rollback architecture that improves safety, repeatability, and protocol correctness of photonic therapy by enforcing cycle-structure compliance before and during optical emission, rather than merely analyzing data after delivery. The disclosed architecture is therefore directed to a specific real-time hardware-and-firmware control architecture for a physical optical emitter, in which compliance evaluation is coupled to a hardware-level emission interlock that physically interrupts continued optical emission upon non-compliance; the disclosed architecture is not directed to performing computations in the abstract, to a mental process, or to mere collection or presentation of data, and the recited audit logic produces a concrete physical effect on the emitter and on the tissue interface in real time.

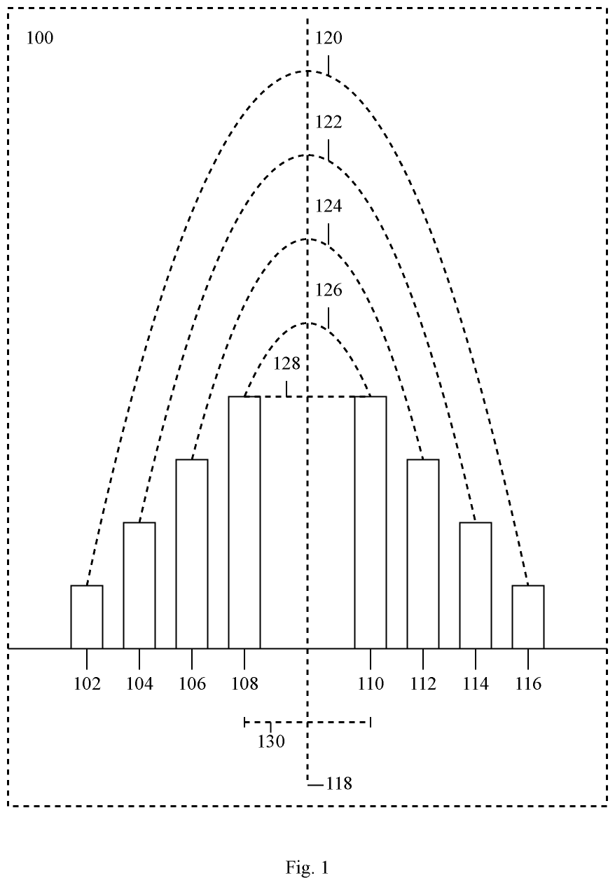

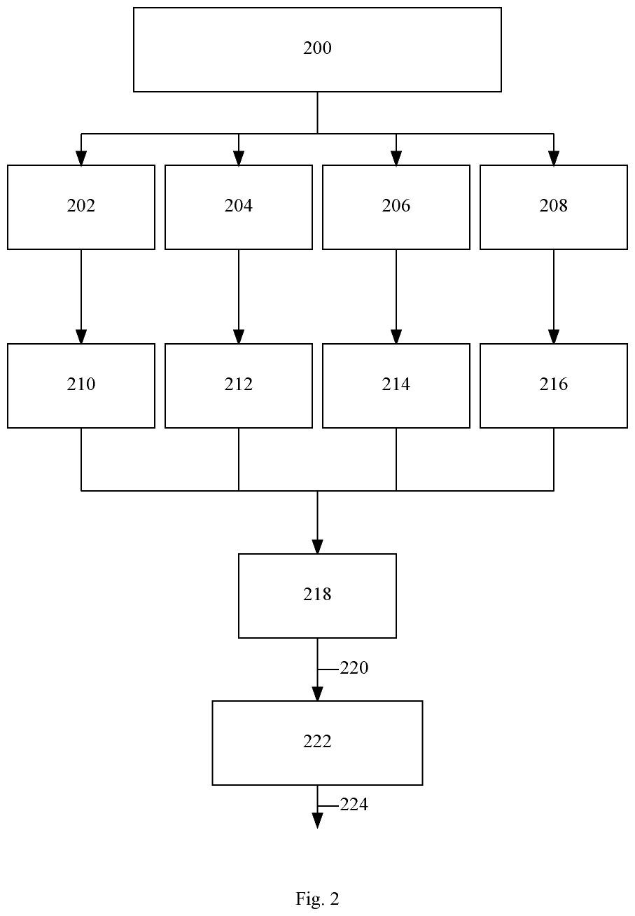

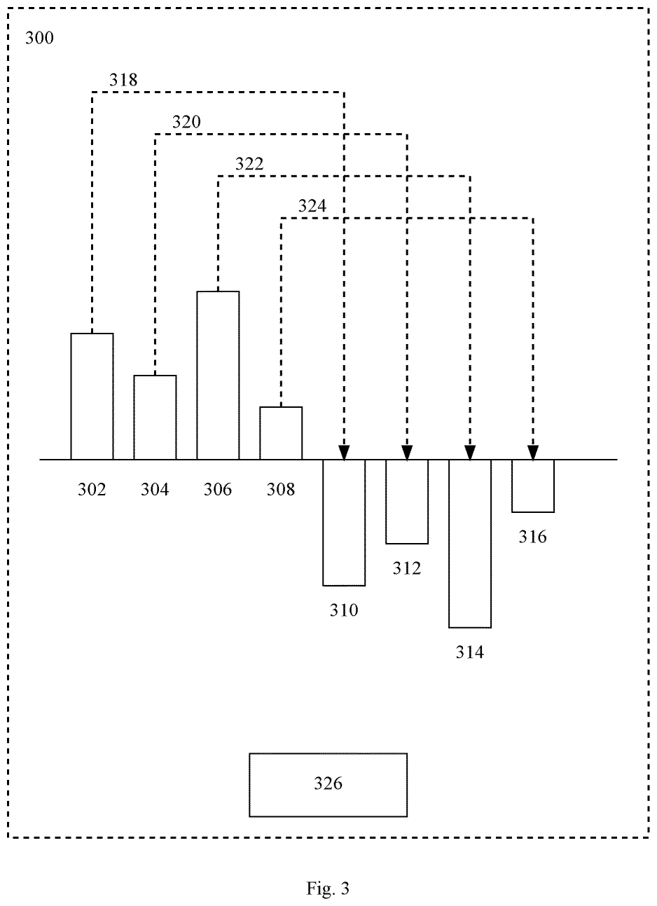

As used herein, a cycle refers to one eight-sub-pulse burst event such as burst 100. A sub-pulse refers to a temporally discrete optical emission segment within such a cycle, including by way of example the first sub-pulse 102, second sub-pulse 104, third sub-pulse 106, fourth sub-pulse 108, fifth sub-pulse 110, sixth sub-pulse 112, seventh sub-pulse 114, and eighth sub-pulse 116 shown in FIG. 1. A mirror pair refers to a paired relationship between sub-pulse positions n and 9-n, including the first mirror pair 120, second mirror pair 122, third mirror pair 124, and fourth mirror pair 126 shown in FIG. 1. A dimensionless J-cost symmetry residual refers to the non-energy, dimensionless quantity derived from applying the disclosed J-cost functional to amplitude ratios of mirror-paired sub-pulses, as computed using the audit flow of FIG. 2. A signed eight-slot ledger schedule refers to a cycle-indexed set of signed values satisfying equal-and-opposite mirror relations, such as the signed schedule 300 shown in FIG. 3. Ledger-neutral refers to satisfaction of the cycle neutrality condition in which the signed schedule sums to zero across the cycle and, in the disclosed formalism, corresponds to zero net cost across the valid ledger window. Non-thermal refers to delivery constrained such that temperature rise at the tissue interface remains below a maximum temperature-rise limit T_max during a treatment session, where T_max is less than about 0.3 C in a preferred embodiment and not more than about 1.0 C in a broader embodiment. Biological tissue refers to tissue to which the disclosed exact eight-sub-pulse audited photonic architecture is applied and can include dermal tissue, wound-associated tissue, joint-associated tissue, superficial vascular tissue targets, and veterinary tissue targets, with dermal tissue, wound-associated tissue, and joint-associated tissue being preferred target classes. Audit pass refers to satisfaction of symmetry, ledger, and, where applicable, additional dose, temperature, calibration, and safety thresholds. Rollback refers to an emission-disable event, power-rail interruption, or other forced interruption of continued emission triggered upon protocol non-compliance. For the avoidance of doubt, the subscript expression 9-n in I_9-n and phi_9-n denotes mirror-pair position arithmetic within the eight-sub-pulse cycle, and is not subtraction performed on a variable named I or phi; accordingly, when n=1 the mirror-paired position is 8, when n=2 the mirror-paired position is 7, when n=3 the mirror-paired position is 6, and when n=4 the mirror-paired position is 5.

The disclosed architecture uses selected Recognition Science support points, but only to the extent tied to the present photonic therapy architecture and not to import the full Recognition Science framework. The disclosed J-cost functional used by the architecture is J(x)=½(x+1/x)−1. The disclosed architecture uses an exact eight-part cycle structure and associated phase-cancellation support as technical grounding for the exact cycle form stated herein. The disclosed coherence quantum is E_coh=phi\hat{ }−5, and the exponent 5 is disclosed as forced by the stated conditions of the framework used herein. The disclosed substrate-support rationale includes overlap of E_coh with water hydrogen-bond and protein hydrogen-bond bands together with disclosed water dynamical support. These support points matter in the present disclosure because they support the exact pulse architecture, the non-arbitrary preferred threshold package, the non-arbitrary preferred carrier-selection package, and the machine-verified viability package used to justify the preferred implementation. These support points are therefore architectural support anchors for the disclosed therapy architecture rather than abstract theory presented for its own sake.

With reference to FIG. 1, burst 100 defines the disclosed core pulse architecture. Burst 100 contains exactly eight sub-pulses 102, 104, 106, 108, 110, 112, 114, and 116 arranged symmetrically about mirror midpoint 118. Sub-pulses 102-116 are not an arbitrary illustrative pulse count. Rather, the exact eight-sub-pulse cycle is the disclosed, non-arbitrary, and load-bearing cycle structure around which the preferred system, method, timing, carrier, and control architecture are built. The sub-pulses are indexed n=1 through 8 and are grouped into mirror pairs 120, 122, 124, and 126 corresponding respectively to positions (1,8), (2,7), (3,6), and (4,5). In the disclosed mirror architecture, the pulse intensities satisfy the intensity mirror relation identified by label 128, namely I_n=I_9-n, and the pulse phases satisfy the phase mirror relation identified by label 130, namely phi_n=phi_9-n+pi. The disclosed cycle structure therefore requires both mirror-pair organization across midpoint 118 and the stated intensity and phase relations for the disclosed preferred architecture.

In operation, burst 100 can be generated in response to desired treatment session parameters, target tissue designation, device calibration state, stored or generated waveform template information, photon-count limits, temperature limits, an applicable mismatch tolerance, and an applicable residual threshold. The result is an eight-sub-pulse waveform that either passes compliance and is delivered to tissue or fails compliance and is blocked, inhibited, paused, terminated, temporarily shut down, latched shut down, reset-restricted, or rolled back. In a broad fallback position, the architecture preserves exact mirror equality as the governing relation for the pulse pairs of burst 100. In another fallback position, the pulse pairs may be required to satisfy mirror compliance within a predetermined mismatch tolerance. In a narrower preferred fallback position, the mismatch tolerance is within plus or minus 0.5 percent. In the preferred implementation, however, exact mirror equality remains the clearest architectural statement because exact mirror equality yields exact zero residual under the disclosed audit logic.

FIG. 2 depicts an audit input set 200 feeding first, second, third, and fourth pair-ratio calculators 202, 204, 206, and 208. The outputs of pair-ratio calculators 202-208 are provided respectively to first, second, third, and fourth J evaluators 210, 212, 214, and 216. The outputs of J evaluators 210-216 are summed by residual summer 218 to produce residual output 220 corresponding to the symmetry residual R_sym, and threshold comparator 222 evaluates residual output 220 to produce pass/fail output 224. In preferred embodiments, audit input set 200 includes commanded pulse values, measured pulse values, or both. In preferred implementations where direct measurement is available, measured values govern acceptance and fault handling, while commanded values are preserved for waveform generation, diagnostics, calibration analysis, and commanded-versus-measured deviation logging. In fallback branches where direct measurement is unavailable, commanded values, model-based values, or other available compliance inputs can be used, with direct measurement remaining the preferred acceptance branch.

The disclosed J-audit residual evaluated using FIG. 2 is R_sym=J(I_1/I_8)+J(I_2/I_7)+J(I_3/I_6)+J(I_4/I_5), where J(x)=½(x+1/x)−1. J(x) is the disclosed unique cost functional used in the architecture disclosed herein. Because J(x) is non-negative for x greater than zero and J(x) equals zero if and only if x equals one, exact mirror equality for all four pulse pairs yields exact zero residual. Accordingly, where sub-pulse 102 matches sub-pulse 116, sub-pulse 104 matches sub-pulse 114, sub-pulse 106 matches sub-pulse 112, and sub-pulse 108 matches sub-pulse 110 according to the disclosed mirror relation, the corresponding residual output 220 is exactly zero. The residual output 220 is dimensionless and does not have units of joules. Threshold comparator 222 therefore compares residual output 220 to a dimensionless threshold rather than to an energy threshold.

The workflow associated with FIG. 2 can proceed by acquiring commanded pulse values, acquiring measured pulse values where direct sensing is available, acquiring photon-count, optical-intensity, or detector-based verification inputs where applicable, computing pair ratios using measured values for acceptance and fault handling in preferred embodiments, using commanded values, model-based values, or other available compliance inputs only in fallback branches where direct measurement is unavailable, applying J to each pair ratio using J evaluators 210, 212, 214, and 216, summing pair penalties using residual summer 218, comparing the aggregate residual using threshold comparator 222, emitting pass/fail output 224, and storing diagnostic information including threshold margin and commanded-versus-measured deviations where applicable. In this way, FIG. 2 provides both a compliance architecture and a make-and-use architecture for the symmetry audit.

In a broad fallback position, pass/fail output 224 is based on a predetermined residual threshold. In a narrower fallback position, pass/fail output 224 can depend on both a predetermined mismatch tolerance and a predetermined residual threshold. In a preferred threshold package, the mirror mismatch is within plus or minus 0.5 percent and the residual threshold is R_sym less than or equal to 1/20000. In a narrower exact preferred implementation, the single-pair penalty at the plus or minus 0.5 percent mismatch boundary is 1/80400 and the four-pair aggregate residual is 1/20100, which is strictly less than 1/20000. These exact values are exact, proof-backed support anchors for the preferred threshold regime and show that the preferred operating point is not arbitrary. These values support the preferred implementation package.

FIG. 3 depicts signed schedule 300 having slots 302, 304, 306, 308, 310, 312, 314, and 316 together with first, second, third, and fourth anti-mirror relations 318, 320, 322, and 324 and zero-sum output 326. In the preferred implementation, signed schedule 300 satisfies delta_0=−delta_4, delta_1=−delta_5, delta_2=−delta_6, and delta_3=−delta_7, thereby yielding the cycle neutrality condition in which the sum from k=0 to 7 of delta_k equals zero. The anti-mirror relations represented by 318, 320, 322, and 324 therefore provide an independent signed neutrality architecture across slots 302-316. Zero-sum output 326 indicates that the preferred schedule form is ledger-neutral across the cycle.

Signed schedule 300 is independent from the J-audit of FIG. 2. The J-audit addresses amplitude-ratio precision through a dimensionless residual, while signed schedule 300 addresses equal-and-opposite signed balancing across the cycle. These two guarantees are distinct, independent, complementary, and non-interchangeable. A burst can satisfy the residual logic of FIG. 2 while failing the signed neutrality of FIG. 3, or can satisfy the signed neutrality of FIG. 3 while failing the residual logic of FIG. 2. Continued emission in the disclosed architecture requires both conditions to pass, subject further to applicable safety conditions. The architecture is therefore not merely a balanced pulse system in a generic sense, but a dual-guarantee architecture with separately defined and separately evaluated compliance conditions.

In operation, signed schedule 300 can be stored, generated, or otherwise established from session parameters before or during treatment. Validation of signed schedule 300 can include checking the equal-and-opposite relations represented by anti-mirror relations 318, 320, 322, and 324, optionally computing the cycle sum represented by zero-sum output 326, and producing a pass/fail neutrality state for subsequent control logic. In a broad fallback position, the signed schedule may satisfy a predetermined neutrality condition. In a narrower fallback position, the signed schedule satisfies equal-and-opposite mirror relations. In the preferred position, delta_k=−delta_k+4 for k=0 through 3. The preferred signed-schedule form is proof-backed, non-arbitrary, and architecture-defining for the preferred implementation package and is not merely a convenient mathematical representation.

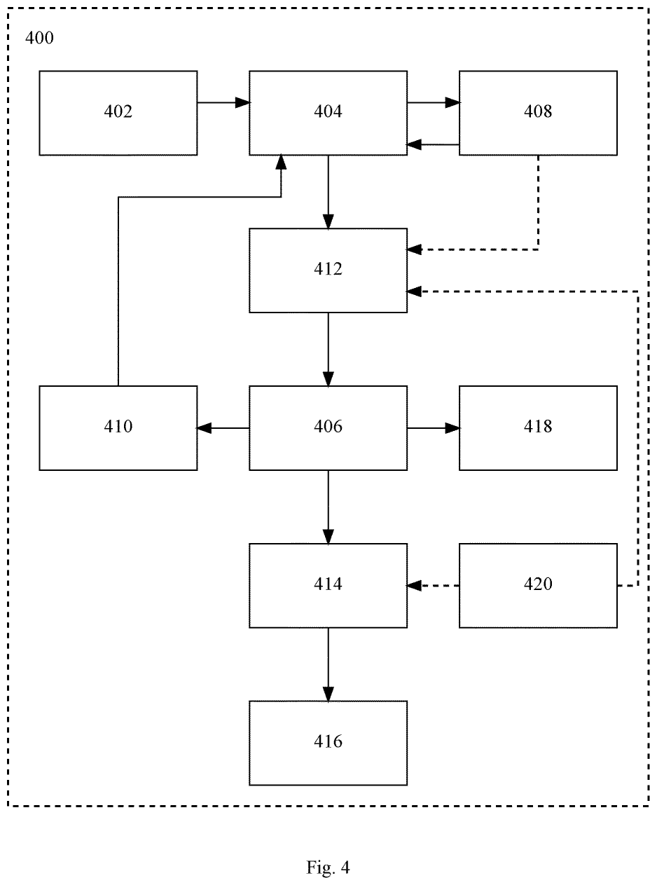

FIG. 4 depicts overall system 400 including clock module 402, controller 404, emitter array 406, verifier 408, photon feedback subsystem 410, rollback gate 412, coupling interface 414, target tissue 416, telemetry output 418, and power subsystem 420. Clock module 402 provides the timing reference used to synchronize pulse generation, verification operations, signed-schedule auditing, treatment sequencing, and, where applicable, calibration-state evaluation. Controller 404 generates or retrieves the exact eight-sub-pulse waveform, applies the mirror architecture, receives verification results, and determines whether emission should continue. Emitter array 406 produces the optical sub-pulses directed through coupling interface 414 toward target tissue 416. Verifier 408 computes symmetry compliance, residual compliance, signed-schedule compliance, and other applicable conditions. Photon feedback subsystem 410 measures one or more optical output properties, preferably including photon count and, where applicable, optical intensity or detector-based pulse verification. Rollback gate 412 interrupts emission when a non-compliance condition occurs. Telemetry output 418 produces an audit record for storage or transmission. Power subsystem 420 supplies operating power to the active components of system 400.

Coupling interface 414 preserves optical coupling, thermal buffering, and biocompatibility between emitter array 406 and target tissue 416. In preferred embodiments, coupling interface 414 is implemented as a hydrogel interface and may include a silver-gel hydrogel formulation. In broader embodiments, coupling interface 414 can be any coupling structure that preserves optical coupling, non-thermal delivery, and suitable biocompatibility for the intended tissue interface. Target tissue 416 can include dermal tissue, wound-associated tissue, joint-associated tissue, superficial vascular tissue targets, and veterinary tissue targets, with dermal tissue, wound-associated tissue, and joint-associated tissue being preferred target classes and with superficial vascular and veterinary targets preserved as narrower or downstream target classes rather than the main make-and-use emphasis of the disclosure.

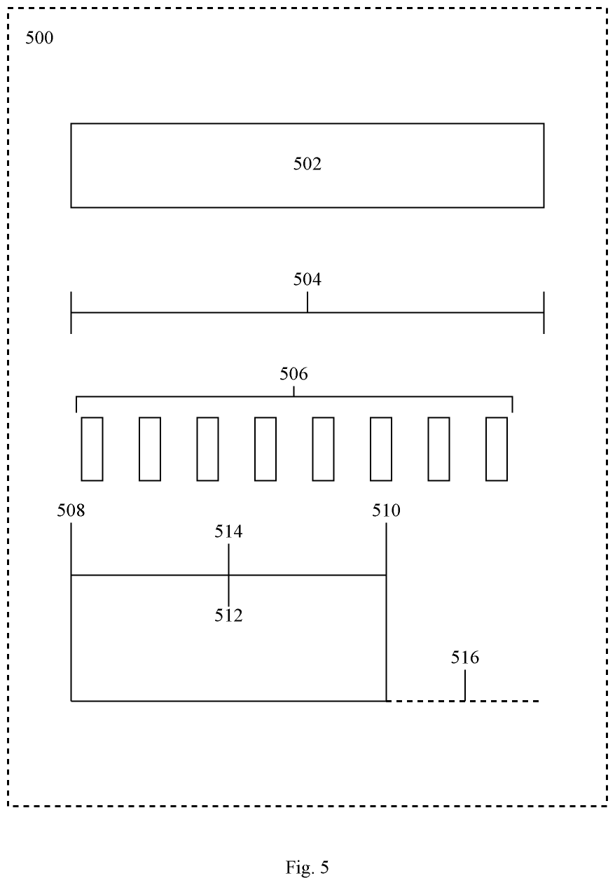

FIG. 5 depicts timing architecture 500 including master clock 502, cycle period 504, sub-pulse sequence 506, session start 508, session end 510, preferred cycle count 512, active emission interval 514, and optional repeat interval 516. In a preferred implementation, master clock 502 is about 125 Hz and is generated from a dual-cavity phi-clock timing reference. Cycle period 504 is therefore about 8.0 ms as the reciprocal of the 125 Hz master frequency, and sub-pulse sequence 506 defines the corresponding eight-sub-pulse pattern within each cycle. In a preferred implementation, the sub-burst rate is about 1000 Hz, corresponding to eight sub-pulses delivered within each 125 Hz cycle. Preferred cycle count 512 is about 200 cycles, yielding active emission interval 514 of about 1.6 seconds from the relation 200/125=1.6 s. Optional repeat interval 516 may represent a repeated treatment schedule, such as repeat treatment after a prior audit pass and an intervening time interval, including in some embodiments an approximately 24-hour repeat interval.

Timing architecture 500 synchronizes the pulse generator, verifier, ledger auditor, treatment sequencer, and, where applicable, calibration-state evaluation. In broad fallback positions, a treatment session may comprise one or more cycles. In narrower fallback positions, a treatment session may comprise a plurality of cycles. In the preferred fallback position, preferred cycle count 512 is about 200 cycles. Timing faults, drift faults, synchronization faults, and other time-base faults can independently trigger pause, inhibition, temporary shutdown, latched shutdown, termination, or rollback where applicable. The preferred timing package represented by FIG. 5 is proof-backed, non-arbitrary, and architecture-defining for the preferred implementation package rather than a casual operating example.

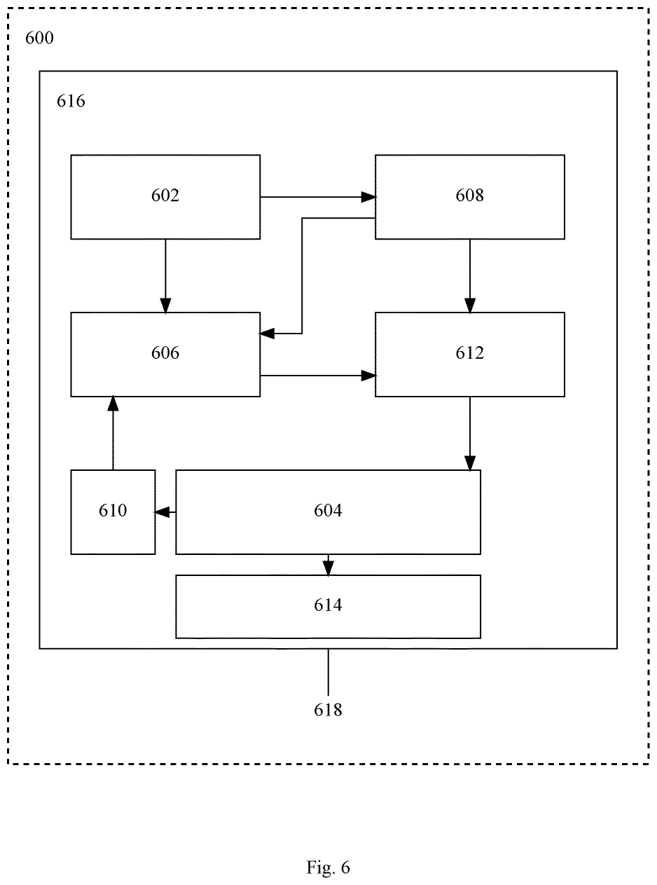

Continuing with FIG. 6, preferred device embodiment 600 includes phi-clock oscillator 602, GaN micro-LED array 604, FPGA verifier 606, microcontroller 608, PIN-diode set 610, rollback circuitry 612, hydrogel pad 614, enclosure 616, and patient-contact side 618. Phi-clock oscillator 602 provides a preferred timing reference for the exact eight-sub-pulse architecture, can be configured as a dual-cavity phi-clock oscillator, and can generate or stabilize the timing basis corresponding to master clock 502 of FIG. 5. GaN micro-LED array 604 is the preferred emitter implementation and, in preferred embodiments, is implemented as an 8×8 array operating at about 450+/−20 nm. FPGA verifier 606 provides a preferred hardware-level implementation of one or more verifier functions, including fast symmetry checking, residual evaluation support, and fault gating, and in preferred implementations operates with less than about 100 ns verification latency. Microcontroller 608 provides higher-level treatment sequencing, session control, logging, parameter management, fault coordination, and communication control. PIN-diode set 610 provides preferred direct sensing of emitted optical output, including photon-count verification and, where applicable, optical intensity or detector-based pulse verification, and can include multiple PIN photodiodes for per-sub-pulse, per-pair, per-cycle, or per-session measurement. Rollback circuitry 612 forcibly interrupts emission when non-compliance is detected. Hydrogel pad 614 forms the preferred tissue coupling interface and can be implemented as a silver-gel hydrogel delivery pad. Enclosure 616 houses the active and passive device components, and patient-contact side 618 identifies the side intended to face the treatment site.

In preferred embodiments, GaN micro-LED array 604 is individually addressable through DAC-based drive control and can be controlled using 12-bit DAC addressing. In preferred embodiments, array 604 is a principal supported implementation class for the disclosed architecture because an individually addressable or grouped-emitter implementation naturally supports generation of the exact eight-sub-pulse cycle, preservation of the disclosed mirror-pair architecture, and compliance evaluation under the disclosed J-audit and signed-schedule logic. Grouped-emitter arrays are also supported as a principal implementation class where the grouped arrangement still generates the exact eight-sub-pulse cycle and preserves the disclosed compliance criteria. A single-source modulated emitter arrangement is not treated as a co-equal implementation class and should be preserved only where supported with concrete operational detail showing how the same exact-cycle and compliance conditions are satisfied. If such concrete support is not implemented in a given embodiment, the single-source branch is omitted rather than generalized.

In preferred embodiments, PIN-diode set 610 includes one or more optical detectors positioned to measure emitted light for per-sub-pulse, per-pair, per-cycle, or per-session verification. Preferred direct sensing modalities therefore include photon-count sensing, optical-intensity sensing, and detector-based pulse verification. Where implemented, such direct sensing modalities govern acceptance and fault handling. In fallback branches where direct sensing is unavailable, commanded values, model-based values, or other available compliance inputs can be used for evaluation, with direct sensing remaining the preferred acceptance branch. Hydrogel pad 614 can be implemented as a transparent hydrogel, a thermally buffering hydrogel, a sterile single-use hydrogel, or a silver-gel hydrogel. In preferred embodiments, hydrogel pad 614 includes silver loading and is configured to preserve optical coupling, thermal buffering, and biocompatibility at patient-contact side 618.

Rollback circuitry 612 can include a switch, gate, interrupt path, power-rail isolation path, discharge path, or combinations thereof. In preferred embodiments, rollback circuitry 612 includes a rollback gate controlled by verifier logic and is configured to disconnect within about 200 ns after fault detection, and a bleeder or discharge path associated with rollback circuitry 612 can discharge stored energy within about 50 us. These values are preferred implementation anchors and support a strong fault-response posture for the preferred hardware package. In broader embodiments, rollback circuitry 612 can implement any forced interruption architecture that preserves the disclosed control functions of emission interruption, disablement, or rollback upon non-compliance.

Clock module 402 of FIG. 4 can therefore be embodied as phi-clock oscillator 602. Emitter array 406 of FIG. 4 can therefore be embodied as GaN micro-LED array 604. Verifier 408 of FIG. 4 can therefore be embodied in whole or in part by FPGA verifier 606, microcontroller 608, or a combination thereof. Photon feedback subsystem 410 of FIG. 4 can therefore be embodied as PIN-diode set 610 and associated signal-processing circuitry. Rollback gate 412 of FIG. 4 can therefore be embodied as rollback circuitry 612. Coupling interface 414 of FIG. 4 can therefore be embodied as hydrogel pad 614 positioned at patient-contact side 618.

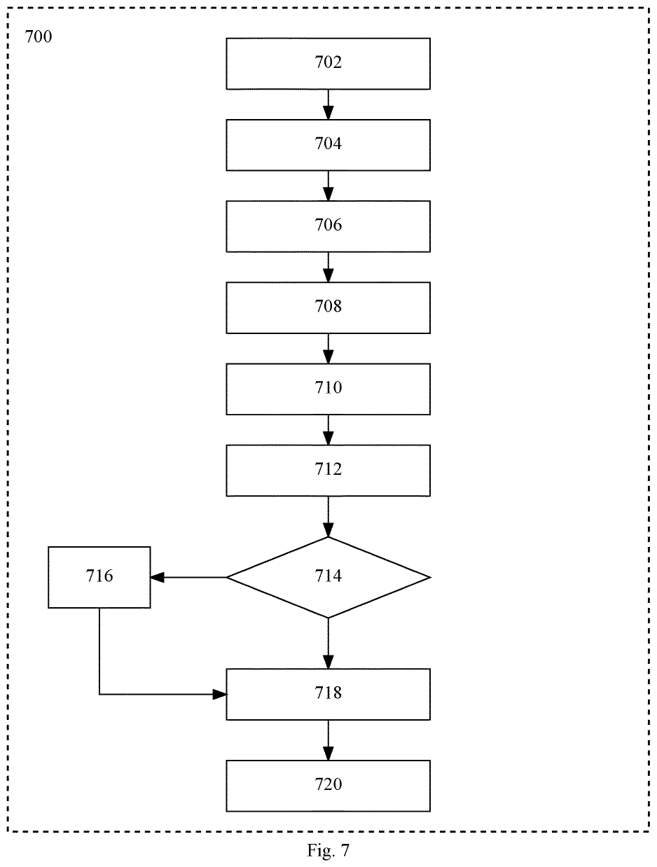

FIG. 7 depicts control workflow 700 including session initiation 702, waveform generation stage 704, symmetry check stage 706, J-audit stage 708, ledger check stage 710, dose and thermal check stage 712, emission continue decision 714, rollback decision 716, logging stage 718, and session completion 720. Session initiation 702 receives a session command, verifies the availability of required treatment parameters, and, in preferred embodiments, confirms that the system is in a valid calibration state. Waveform generation stage 704 loads or generates the exact eight-sub-pulse waveform for burst 100, including the placement of sub-pulses 102-116 and the mirror-pair relations 120-126. Symmetry check stage 706 evaluates whether the pulse pairs satisfy the applicable mismatch tolerance. J-audit stage 708 evaluates the dimensionless symmetry residual corresponding to residual output 220 of FIG. 2. Ledger check stage 710 verifies the neutrality condition of signed schedule 300 of FIG. 3. Dose and thermal check stage 712 evaluates applicable photon-budget and temperature limits. Emission continue decision 714 allows continued emission only while required compliance conditions remain satisfied. Rollback decision 716 places the system into one or more fault outcomes when a required condition fails. Logging stage 718 stores or transmits session and fault information, and session completion 720 records either normal completion or fault termination.

Control workflow 700 can be implemented in hardware logic, firmware, or a combination thereof. This preserves support for control-location fallback positions before narrowing to a specific FPGA-plus-microcontroller implementation. In a hardware-heavy implementation, FPGA verifier 606 may perform fast pass/fail checking and interlocking, while microcontroller 608 performs sequencing, session control, and logging. In a firmware-heavy implementation, microcontroller 608 may perform a greater share of ratio evaluation, residual computation, signed-schedule verification, and fault hierarchy decisions. In a combined implementation, FPGA verifier 606 and microcontroller 608 divide these roles according to latency, safety, and architecture preferences.

Symmetry check stage 706 can receive commanded pulse intensities, measured pulse intensities where direct measurement is available, photon-count inputs, optical-intensity inputs, detector-based verification inputs, and an applicable mismatch tolerance. Symmetry check stage 706 can compute pair ratios or direct mismatch values for the mirror pairs corresponding to 120, 122, 124, and 126, determine whether each mirror pair satisfies the applicable mismatch tolerance, generate a per-pair pass/fail state, and produce an aggregate symmetry state. In a broad fallback position, symmetry check stage 706 can evaluate compliance against a predetermined mismatch tolerance. In a narrower preferred fallback position, the mismatch tolerance is within plus or minus 0.5 percent.

J-audit stage 708 can receive pair ratios derived from measured values for acceptance and fault handling in preferred embodiments and can, in fallback branches, receive pair ratios derived from commanded values, model-based values, or other available compliance inputs where direct measurement is unavailable. J-audit stage 708 computes J of each pair ratio, sums pair penalties, compares aggregate residual to the applicable threshold, and stores residual margin relative to threshold. The output of J-audit stage 708 can therefore correspond to residual value, threshold margin, and pass/fail state. In a broad fallback position, J-audit stage 708 evaluates a predetermined residual threshold. In a preferred threshold position, the threshold is 1/20000. Ledger check stage 710 receives the signed schedule values corresponding to schedule 300, verifies the equal-and-opposite relations corresponding to anti-mirror relations 318, 320, 322, and 324, optionally computes the cycle sum corresponding to zero-sum output 326, and produces a pass/fail ledger-neutrality state and logged diagnostic state. Treatment sequencer functions distributed across session initiation 702, waveform generation stage 704, emission continue decision 714, and session completion 720 receive a session command, cycle count, timing reference, and waveform template, deliver the exact eight sub-pulses for each cycle, apply the cycle count and timing rules represented by timing architecture 500, and gate continued delivery on compliance signals received from stages 706, 708, 710, and 712.

Rollback decision 716 can receive one or more fault causes including symmetry failure, residual-threshold failure, signed-schedule failure, photon-budget exceedance, temperature-limit exceedance, calibration-invalid state, watchdog fault, or other hardware or control faults. Rollback decision 716 can apply fault precedence rules and place the system into one or more control outcomes including temporary emission pause, inhibited emission, terminated emission, rollback, temporary shutdown, latched shutdown, or reset-required shutdown. Rollback decision 716 can further store fault cause, require clearance or reset, and require revalidation before resumed operation where applicable. Logging stage 718 can receive residuals, pair-ratio values, signed-schedule status, photon counts, temperature values, device serial information, timestamps, session outcome, and fault causes, and can store, hash, sign, or transmit the resulting audit record.

A calibration-state manager can be implemented as part of microcontroller 608, FPGA verifier 606, controller 404, or a combination thereof. The calibration-state manager receives clock status, emitter status, sensor status, and reference-check results, determines whether the device is in a valid calibration state, prevents treatment initiation or continuation when the calibration state is invalid, and logs calibration faults. The disclosed architecture therefore treats calibration validity as an independent condition for continued emission rather than as a mere maintenance note.

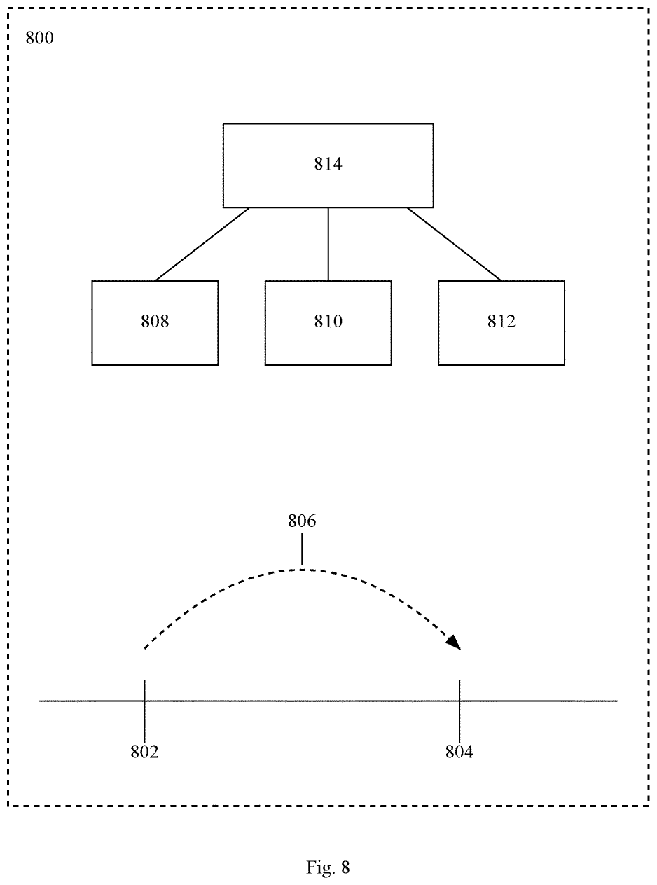

FIG. 8 depicts carrier-support diagram 800 including E_coh marker 802, carrier-energy marker 804, harmonic relation indicator 806, water H-bond band 808, protein H-bond band 810, water transparency window 812, and substrate-support output 814. E_coh marker 802 corresponds to the disclosed coherence quantum E_coh=phi\hat{ }−5. The exponent 5 is disclosed as structurally forced and is not merely background theory. Rather, the forced exponent is a disclosed, load-bearing support point for the preferred carrier-selection logic used in the invention. Carrier-energy marker 804 corresponds in preferred embodiments to a visible carrier around 450+/−20 nm. Harmonic relation indicator 806 corresponds to the preferred harmonic relation abs(E_photon/E_coh_display−31)/31<0.05. Water H-bond band 808 and protein H-bond band 810 represent overlap support between the disclosed coherence quantum and biologically relevant hydrogen-bond energy bands. Water transparency window 812 represents a preferred visible-water transmission region relevant to carrier selection. Substrate-support output 814 represents the resulting support for the preferred carrier package.

The preferred 450+/−20 nm carrier is therefore a proof-backed preferred implementation of the disclosed carrier-selection rule rather than an arbitrary wavelength choice. In a broad fallback position, the carrier may be selected relative to the disclosed coherence-quantum criterion. In a narrower fallback position, the carrier may satisfy a predetermined harmonic-proximity criterion relative to E_coh, a predetermined energy-band criterion relative to E_coh, or both. In preferred implementations, the predetermined harmonic-proximity criterion is satisfied when abs(E_photon/E_coh−N)/N is less than or equal to about 0.05 for some integer N, and the predetermined energy-band criterion is satisfied when E_photon falls within a tolerance band of about plus or minus 5 percent around an integer multiple of E_coh, with the integer N=31 representing one preferred and proof-backed selection rather than a universal requirement. In a further narrower fallback position, the carrier may be a visible carrier selected within water transparency window 812 and relative to the disclosed coherence-quantum criterion. In a carrier implementation-class fallback, the carrier may be implemented using visible solid-state emitter implementations selected under the foregoing criteria. In narrower hardware-class fallbacks, the emitter implementation may be micro-LED-based or GaN-based. In the preferred implementation, carrier-energy marker 804 corresponds to a carrier around 450+/−20 nm and within the disclosed preferred tolerance band of the 31st multiple of E_coh.

The present disclosure also preserves substrate-support rationale tied to FIG. 8. Water H-bond band 808 provides support for overlap between the disclosed coherence quantum and water hydrogen-bond energies. Protein H-bond band 810 provides support for overlap between the disclosed coherence quantum and protein hydrogen-bond energies. Water transparency window 812 provides support for visible-carrier transmission through a relevant water window. Substrate-support output 814 therefore reflects the combined support for the preferred carrier package and does not stand alone as a separate inventive center. Alternative carriers are discussed only within these preserved criteria and are not elevated as free-floating generalizations.

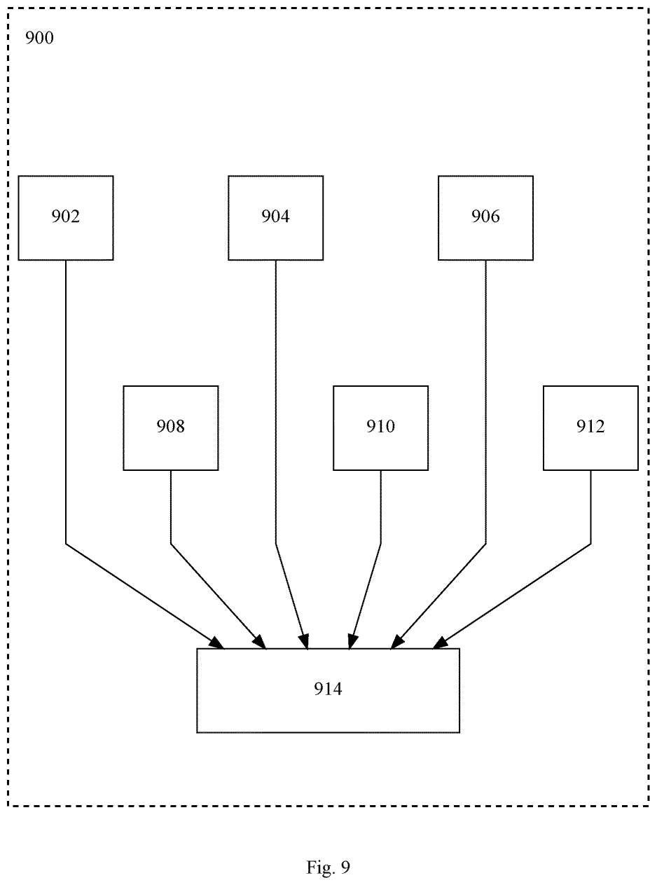

FIG. 9 depicts proof architecture 900 including J-cost support module 902, eight-tick support module 904, coherence exponent module 906, substrate-support module 908, ledger-neutrality module 910, timing and dose support module 912, and master certificate 914. J-cost support module 902 corresponds to proof-backed support for J-cost non-negativity, J(x)=0 iff x=1, exact-zero residual under exact mirror equality, the exact single-pair value J(201/200)=1/80400, and the exact four-pair residual value 1/20100, including support corresponding to mirror_burst_zero_residual, worstCasePairPenalty_value, worstCaseCycleResidual_value, and worstCaseCycleResidual_lt_tolerance. Eight-tick support module 904 corresponds to proof-backed support for the disclosed exact cycle structure, including eight-tick phase cancellation support. Coherence exponent module 906 corresponds to proof-backed support for the exponent in E_coh=phi\hat{ }−5, including ecoh_exponent_forced. Substrate-support module 908 corresponds to proof-backed support for water and protein band overlap, carrier harmonic matching, and water-window placement, including water_substrate_support, protein_substrate_support, and carrier_450 nm_in_band. Ledger-neutrality module 910 corresponds to proof-backed support for equal-and-opposite schedule balancing, signed-schedule neutrality, and ledger neutrality, including mirror_cost_implies_balanced and folding_ledger_neutral. Timing and dose support module 912 corresponds to proof-backed support for preferred timing and photon-count values, including subBurstRate_value, cyclePeriod_value, treatment_200_duration, totalPhotonsPerCycle_value, and treatment_200_photons. Master certificate 914 corresponds to the assembled viability package for the preferred implementation, including photonic_therapy_viable.

In preferred disclosure form, the proof-backed items represented by proof architecture 900 support the preferred cycle structure, the preferred threshold regime, the preferred timing regime, and the preferred carrier regime. These proof-backed items are used throughout the disclosure as technical support for the preferred implementation package. For example, exact-zero residual support helps justify the exact mirror-pair architecture of FIG. 1 and the J-audit logic of FIG. 2. Exact threshold support helps justify the preferred threshold package used in stage 708 of FIG. 7. Coherence exponent and harmonic support help justify the preferred carrier package represented by FIG. 8. Timing and dose support help justify the preferred values represented in FIG. 5 and dose and thermal check stage 712 of FIG. 7.

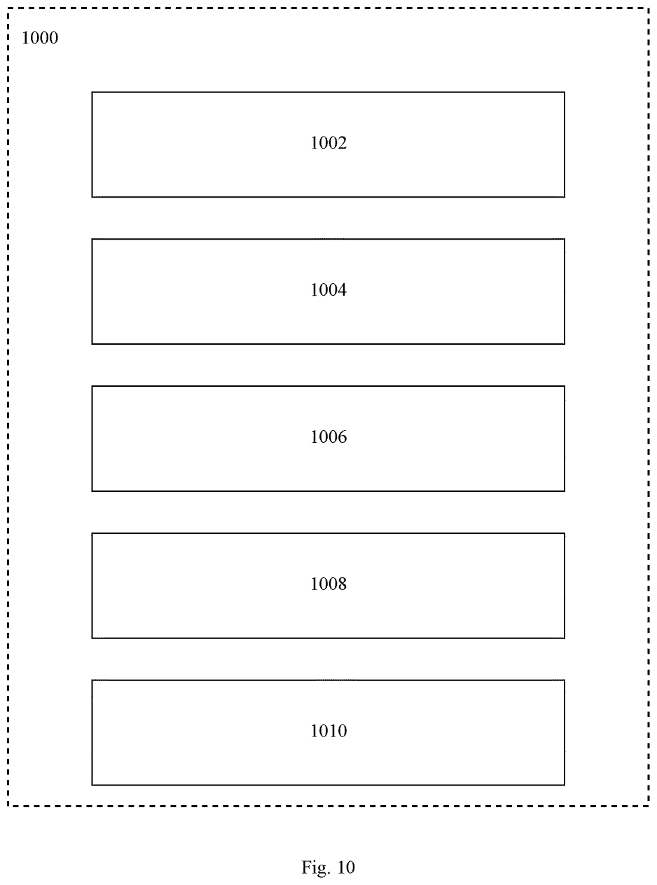

FIG. 10 depicts numerical-example panel 1000 including harmonic-matching panel 1002, residual-threshold panel 1004, photon-budget panel 1006, timing panel 1008, and thermal-budget panel 1010. Harmonic-matching panel 1002 can present the preferred carrier relationship to E_coh by showing the 450 nm photon energy, the nearest integer multiple 31, and a less-than-5-percent deviation relative to the disclosed harmonic criterion represented by harmonic relation indicator 806. Residual-threshold panel 1004 can present the exact preferred values 1/80400, 1/20100, and 1/20000 to illustrate the preferred threshold package and the exact margin by which the worst-in-tolerance four-pair residual remains below the preferred threshold. Photon-budget panel 1006 can present preferred per-sub-pulse, per-cycle, and per-treatment photon limits, including <=1.5×10\hat{ }15 photons per sub-pulse, <=1.2×10\hat{ }16 photons per cycle, and <=2.4×10\hat{ }18 photons for about 200 cycles. Timing panel 1008 can present the preferred timing values associated with master clock 502, cycle period 504, sub-pulse sequence 506, preferred cycle count 512, and active emission interval 514, including the exact derivation from 125 Hz to 8.0 ms, from eight sub-pulses per cycle to a 1000 Hz sub-burst rate, and from 200 cycles at 125 Hz to about 1.6 s active emission. Thermal-budget panel 1010 can present preferred thermal estimates and support for non-thermal operation, including energy-per-sub-pulse, energy-per-cycle, energy-per-treatment, estimated adiabatic temperature rise, and reduction of interface heating through perfusion and hydrogel buffering. These numerical examples do more than compute values. They reinforce why the preferred threshold, timing, carrier, dose, and non-thermal regimes are exact, proof-backed, and non-arbitrary parts of the preferred implementation package.

Photon-budget and temperature control can therefore be implemented using the preferred per-sub-pulse photon cap of less than or equal to 1.5×10\hat{ }15 photons, the preferred per-cycle total of less than or equal to 1.2×10\hat{ }16 photons, and the preferred per-treatment total of less than or equal to 2.4×10\hat{ }18 photons for the preferred about-200-cycle session. Preferred interface temperature rise is less than about 0.3 C, with the coupling medium, including hydrogel pad 614 or coupling interface 414, providing thermal buffering. Exceedance of photon-budget or temperature limits can independently trigger pause, inhibition, temporary shutdown, latched shutdown, termination, or rollback. These values are preferred fallback positions and remain available for dependent claims and for examiner distinction.

A make-and-use workflow can therefore include verifying a valid calibration state, loading or generating the exact eight-sub-pulse waveform template, loading or generating the signed schedule, confirming tissue coupling at coupling interface 414 or hydrogel pad 614 and optional contact sensing, beginning cycle delivery, measuring or verifying mirror-pair intensities, computing pair ratios, computing R_sym, verifying the signed schedule, verifying photon and temperature limits, applying fault precedence rules, continuing emission only while the dimensionless symmetry residual, signed-schedule neutrality condition, photon-budget limit, temperature limit, calibration state, hardware state, and watchdog state remain compliant, temporarily pausing, inhibiting, terminating, temporarily shutting down, latching shut down, or rolling back emission when any required condition fails, storing and optionally transmitting telemetry, and optionally permitting repeat treatment after interval 516 and prior-audit pass. In preferred embodiments, measured values govern acceptance and fault handling when direct measurement is available. Photon-count sensing, optical-intensity sensing, and detector-based pulse verification are preferred direct measurement modalities where implemented. Commanded values remain available for generation and diagnostics, and in the absence of direct measurement, commanded values, model-based values, or other available compliance inputs can be used in a fallback branch.

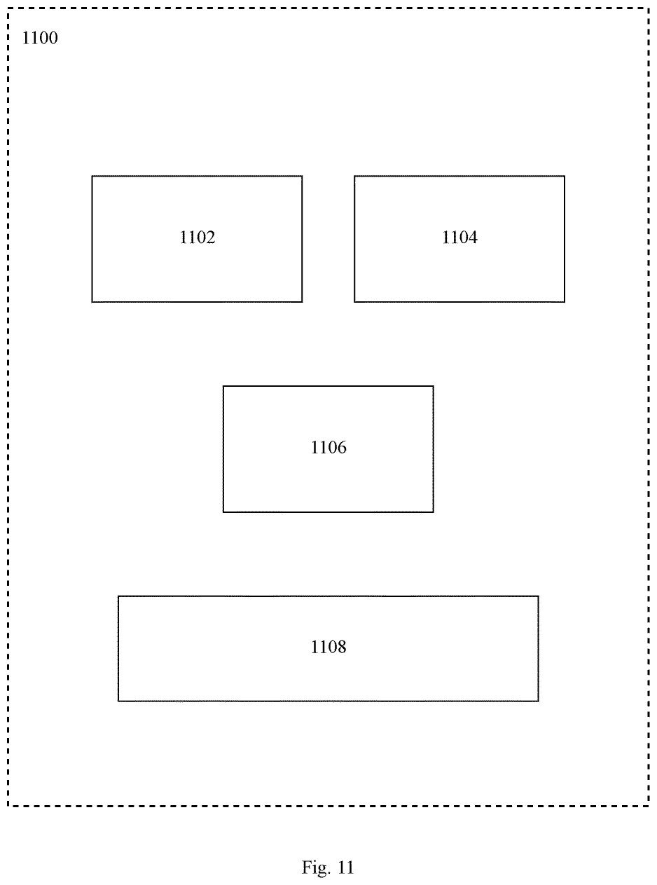

FIG. 11 depicts pilot-data summary 1100 including wound-closure panel 1102, cytokine panel 1104, thermal-observation panel 1106, and preliminary-data notice 1108. Pilot-data summary 1100 is empirical and not machine-verified. Wound-closure panel 1102 can summarize preliminary wound-healing observations obtained using the disclosed exact eight-sub-pulse architecture, including in some pilot data sets approximately 35 percent faster wound closure. Cytokine panel 1104 can summarize preliminary cytokine observations, including IL-6 signal changes where observed and, in some pilot data sets, approximately 28 percent IL- 6 reduction at about 6 hours. Thermal-observation panel 1106 can summarize observed non-thermal behavior, including absence of meaningful thermal artifact or ROS artifact within the tested protocol range. Preliminary-data notice 1108 expressly indicates that the pilot data are preliminary empirical observations and not machine-verified architectural support. Demonstrated validation material such as pilot-data summary 1100 is useful as validation and should not be blended with prophetic examples or with the core inventive identity of the exact audited eight-sub-pulse architecture. The pilot material represented by FIG. 11 can therefore be categorized as demonstrated material rather than as core inventive identity. Demonstrated material can include exact mirror residual logic, preferred threshold arithmetic, timing values, photon-count values, preferred thermal-budget examples, formal machine-verified support, preliminary wound-healing pilot data, and preliminary cytokine pilot data. By contrast, prophetic material can include chaperone-cycle coupling, I kappa B stabilization as a causal mechanism, karyopherin or nuclear-translocation gating, systemic vascular anti-inflammatory use for sepsis or SIRS, collagen-remodeling or procollagen-folding use emphasis unless later separately supported, and broader downstream indication families beyond the demonstrated wound and cytokine observations. The present disclosure therefore maintains a separation between demonstrated material and prophetic material so that validation is presented as validation and not as a substitute for architectural support.

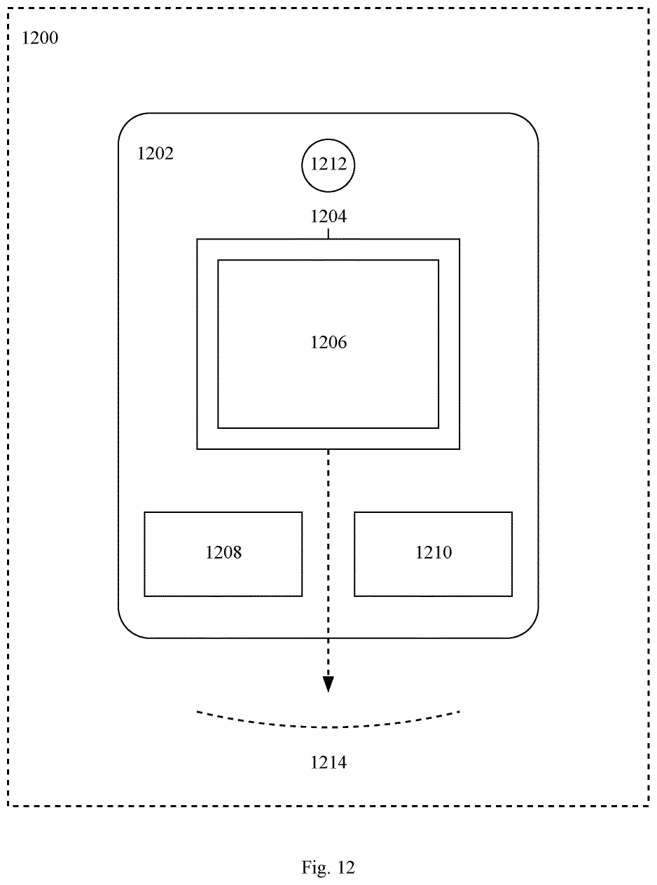

FIG. 12 depicts bandage embodiment 1200 including bandage body 1202, emitter region 1204, hydrogel pad 1206, power source 1208, controller package 1210, status indicator 1212, and wound site 1214. Bandage embodiment 1200 is a preferred downstream embodiment within the patch-type device subclass. Bandage body 1202 supports placement over wound site 1214. Emitter region 1204 delivers the disclosed exact eight-sub-pulse architecture through hydrogel pad 1206 toward wound site 1214. Hydrogel pad 1206 can correspond to or incorporate the functions of hydrogel pad 614 and coupling interface 414 by preserving optical coupling, thermal buffering, and biocompatibility at the treatment interface. Power source 1208 supplies operating power. Controller package 1210 executes or coordinates waveform generation, symmetry verification, residual evaluation, signed-schedule verification, safety logic, and session control. Status indicator 1212 can provide user-visible information such as ready, active, paused, fault, or complete states.

In preferred embodiments, bandage embodiment 1200 is configured as a self-contained wound-healing bandage in which emitter region 1204 is centered over wound site 1214 and hydrogel pad 1206 is placed in direct or substantially direct contact with the treatment site. Power source 1208 can be a battery source sized for one or more treatment sessions. Controller package 1210 can incorporate controller 404, at least part of verifier 408, logging capability corresponding to telemetry output 418, and timing capability corresponding to clock module 402. Status indicator 1212 can present session progress, fault state, or completion state. Bandage embodiment 1200 can be sterile or can include a single-use sterile interface. Although bandage embodiment 1200 is a preferred downstream embodiment, it remains subordinate to the core exact audited eight-sub-pulse architecture and does not redefine the invention.

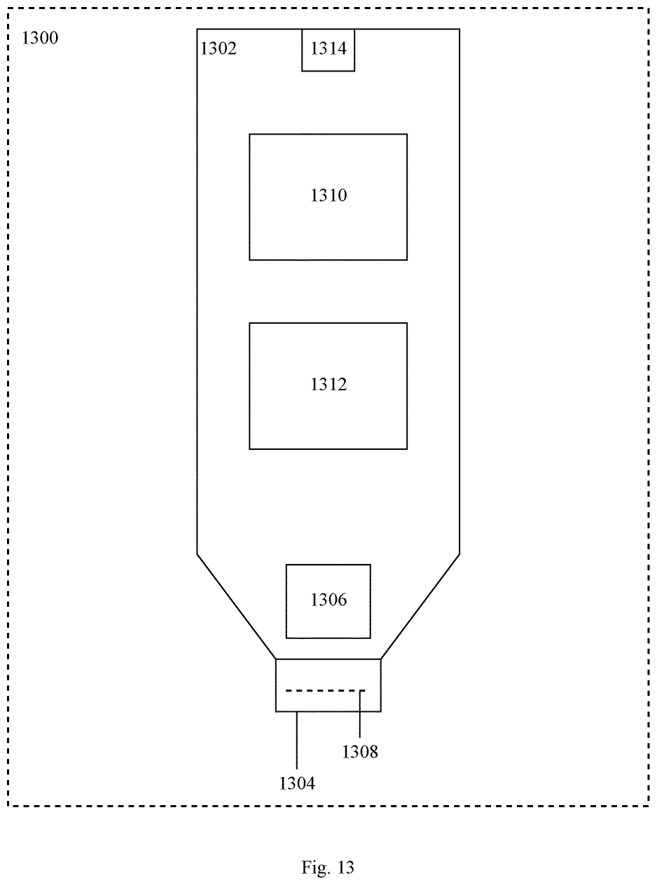

FIG. 13 depicts probe embodiment 1300 including probe housing 1302, treatment tip 1304, emitter array 1306, contact sensor 1308, display 1310, battery 1312, and data or charging port 1314. Probe embodiment 1300 is a preferred downstream embodiment within the handheld-device subclass. Probe housing 1302 supports manual positioning of treatment tip 1304 relative to a selected tissue site. Emitter array 1306 is positioned at or near treatment tip 1304 and delivers the disclosed exact eight-sub-pulse architecture to the selected treatment region. Contact sensor 1308 can be used as an optional interlock or support feature to confirm suitable contact or proximity before continued emission. Display 1310 can present session information, residual status, symmetry status, battery state, fault information, or completion state. Battery 1312 provides portable power. Data or charging port 1314 can support charging, local data export, or both.

In preferred embodiments, probe embodiment 1300 is configured for treatment of joint-associated tissue or other localized treatment sites accessible by a handheld applicator. Contact sensor 1308 may inhibit emission until suitable contact is confirmed. Display 1310 may provide real-time status related to the disclosed audit architecture, such as pass or fail indication, residual information, session state, or battery condition. Data or charging port 1314 may support transfer of telemetry records corresponding to logging stage 718 or may support charging of battery 1312. Probe embodiment 1300 can therefore embody the same exact audited eight-sub-pulse architecture in a handheld format while remaining subordinate to the core architecture rather than becoming a separate inventive center.

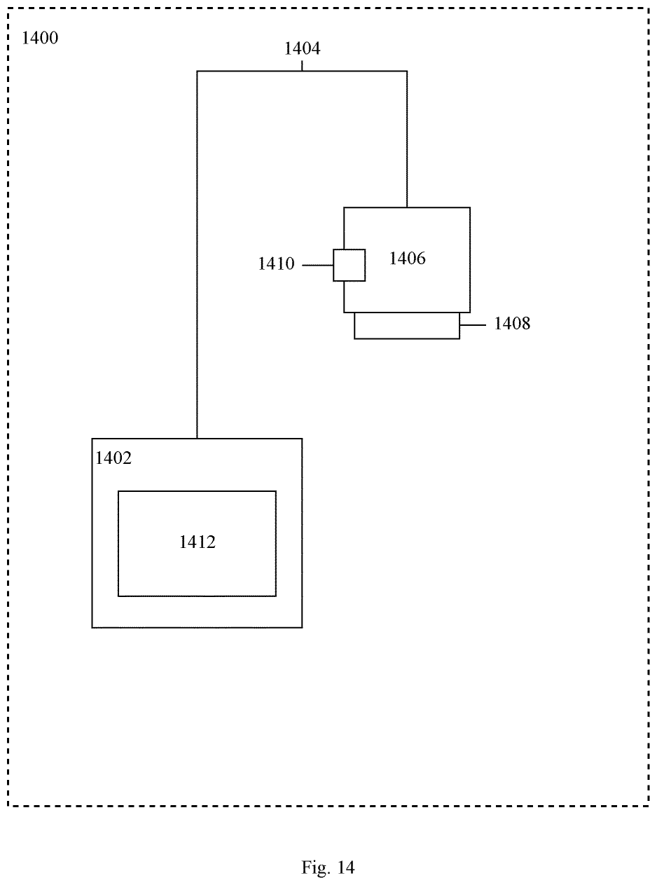

FIG. 14 depicts tabletop embodiment 1400 including base unit 1402, articulated arm 1404, treatment head 1406, enlarged emitter array 1408, thermal monitor 1410, and control console 1412. Tabletop embodiment 1400 is a stationary downstream embodiment and may be described as optional or prophetic depending on the support emphasis of a particular filing strategy. Base unit 1402 supports power, control, thermal-management, and structural functions. Articulated arm 1404 positions treatment head 1406 relative to a treatment site. Enlarged emitter array 1408 can provide a larger-area implementation of emitter array 406 or GaN micro-LED array 604 while still preserving the exact eight-sub-pulse architecture, mirror pairing, residual logic, signed-schedule logic, and non-thermal control framework. Thermal monitor 1410 can provide a supplemental temperature safeguard corresponding to dose and thermal check stage 712. Control console 1412 can provide user configuration, session initiation, display, logging access, or combinations thereof.

Where preserved, tabletop embodiment 1400 can be used for skin-treatment or collagen-related applications, but such downstream use context remains subordinate to the core exact audited architecture and may be categorized as optional or prophetic depending on the level of separate support. Treatment head 1406 and enlarged emitter array 1408 still operate according to the same disclosed cycle structure and compliance architecture used elsewhere in the disclosure. Thermal monitor 1410 can support an additional thermal interlock or confirmation function, and control console 1412 can provide implementation-specific user interface features. FIG. 14 is an optional downstream embodiment figure and may be reduced or removed in a leaner filing posture without destabilizing the core architectural disclosure.

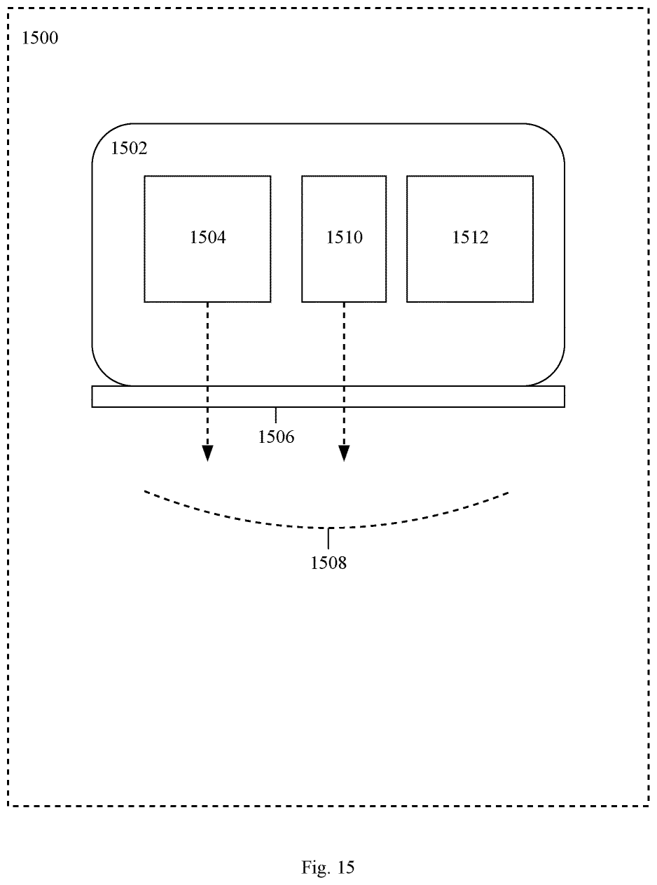

FIG. 15 depicts vascular patch embodiment 1500 including flexible patch body 1502, emitter array 1504, adhesive layer 1506, vascular target region 1508, optional PPG sensor 1510, and controller package 1512. Vascular patch embodiment 1500 is a downstream patch-type embodiment and can be described as prophetic in view of the support emphasis of a particular filing strategy. Flexible patch body 1502 positions emitter array 1504 over vascular target region 1508. Adhesive layer 1506 secures the structure in place. Optional PPG sensor 1510 can assist with vascular alignment, contact confirmation, or monitoring of positioning conditions. Controller package 1512 coordinates operation of the disclosed exact eight-sub-pulse architecture and can incorporate waveform generation, verification logic, and session control functions analogous to controller package 1210 or controller 404.

Where preserved, vascular patch embodiment 1500 can be used in a downstream superficial vascular treatment context, but such context remains subordinate to the core exact audited architecture and should not be allowed to broaden the make-and-use disclosure beyond the concrete implementation detail provided herein. Optional PPG sensor 1510 remains an optional support feature rather than a required element of the core invention. FIG. 15 is therefore an optional downstream embodiment figure and may be reduced or removed without destabilizing the core disclosure, the core drawing set, or the core claim architecture.

The disclosed hardware and control architecture can therefore be packaged in multiple subclasses while preserving the same exact audited eight-sub-pulse architecture. Patch-type devices include embodiments such as bandage embodiment 1200 and, where preserved, vascular patch embodiment 1500. Handheld devices include embodiments such as probe embodiment 1300. Stationary devices include embodiments such as tabletop embodiment 1400. Kits and software-controlled treatment hardware can also be implemented around the same disclosed architecture. A make-and-use workflow can accordingly include verifying a valid calibration state, loading or generating the exact eight-sub-pulse waveform template, loading or generating the signed schedule, confirming tissue coupling at coupling interface 414, hydrogel pad 614, hydrogel pad 1206, treatment tip 1304, treatment head 1406, or adhesive placement over vascular target region 1508 as applicable, beginning cycle delivery, measuring or verifying mirror-pair intensities, computing pair ratios, computing R_sym, verifying the signed schedule, verifying photon and temperature limits, applying fault precedence rules, continuing emission only while the dimensionless symmetry residual, signed-schedule neutrality condition, photon-budget limit, temperature limit, calibration state, hardware state, and watchdog state remain compliant, temporarily pausing, inhibiting, terminating, temporarily shutting down, latching shut down, or rolling back emission when any required condition fails, storing and optionally transmitting telemetry, and optionally permitting repeat treatment after optional repeat interval 516 and prior-audit pass.

Within this workflow, measured values govern acceptance and fault handling in preferred embodiments when direct measurement is available. Photon-count sensing, optical-intensity sensing, and detector-based pulse verification are preferred direct measurement modalities where implemented, including through PIN-diode set 610 or other optical feedback arrangements. Commanded values remain available for waveform generation and diagnostics. In the absence of direct measurement, commanded values, model-based values, or other available compliance inputs can be used in a fallback branch. Any one of symmetry failure, residual-threshold failure, signed-schedule failure, photon overrun, temperature overrun, calibration-invalid state, or watchdog fault can trigger interruption of continued emission. Continued emission, paused emission, inhibited emission, terminated emission, temporary shutdown, latched shutdown, reset-required shutdown, and rollback are all expressly preserved as distinct control framings where supported.

Mechanistic rationale can also be provided, while remaining carefully segregated from the claim-defining identity of the invention. Within that rationale, water-mediated phase alignment can be described as a rationale in which the disclosed exact eight-sub-pulse architecture interacts with water-associated dynamics supported by the disclosed substrate support. Hydrogen-bond energy matching can be described as a rationale in which the preferred carrier package and coherence-quantum support place the disclosed architecture in a biologically relevant energetic context. Ledger-neutral input can be described as a rationale in which the independent signed schedule supports a no-net-bias cycle interpretation under the disclosed formalism. Chaperone coupling can be described as a framework-supported mechanism with empirical verification pending. These rationales may help explain why the architecture is of interest for protein-folding-related contexts.

An anti-inflammatory rationale can likewise be presented in carefully bounded form. Within that rationale, I kappa B stabilization may be described as a mechanistic hypothesis with empirical quantification pending. Nuclear-translocation gating may be described as a hypothesized or preliminary mechanism that may reduce rate in appropriate settings. Non-thermal and ledger-neutral framing may be used to explain why the architecture differs from generic heat-based intervention. These anti-inflammatory rationales are explanatory and can support downstream indication discussions.

Formal machine-verification and proof support can also be expressly tied to the disclosed architecture. Mirror-zero support can justify the exact mirror-pair architecture and the exact-zero condition under exact mirror equality, including named support corresponding to mirror_burst_zero_residual. Preferred-threshold support can justify the preferred threshold sizing and the exact preferred values used in the preferred implementation, including worstCasePairPenalty_value, worstCaseCycleResidual_value, and worstCaseCycleResidual_lt_tolerance. Signed-balance support can justify the equal-and-opposite cycle schedule and the neutrality condition represented by signed schedule 300 and zero-sum output 326, including mirror_cost_implies_balanced and folding_ledger_neutral. Coherence-exponent support can justify the exponent in E_coh=phi\hat{ }−5 and the resulting preferred carrier-selection logic, including ecoh_exponent_forced. Harmonic support can justify the preferred 450+/−20 nm carrier package and the preferred harmonic relation represented by harmonic relation indicator 806, including carrier_450 nm_in_band. Water and protein overlap support can justify the substrate-support framing represented by water H-bond band 808 and protein H-bond band 810, including water_substrate_support and protein_substrate_support. Timing and photon-count support can justify the preferred timing and dose values represented by timing architecture 500 and numerical-example panel 1000, including subBurstRate_value, cyclePeriod_value, treatment_200_duration, totalPhotonsPerCycle_value, and treatment_200_photons. Master certificate 914 can justify the assembled preferred viability package, including photonic_therapy_viable. These proof-backed items support the exact form of the preferred cycle structure, the exact form of the preferred threshold regime, the exact form of the preferred timing regime, and the exact form of the preferred carrier regime.

The disclosure therefore provides a non-thermal photonic therapy architecture with principled dosimetry, rigorous dimensionless audit, dual independent balance guarantees, real-time auditing, rollback safety, water and protein substrate grounding, machine-verification support, and an immutable or reconstructable audit trail. Those advantages arise from the disclosed exact eight-sub-pulse architecture, its dimensionless J-audit, its independent signed ledger schedule, and its real-time compliance enforcement rather than from any one downstream packaging format, pilot result, or mechanistic narrative.

The disclosed architecture supports treatment methods in which the exact eight-sub-pulse cycle is generated, the disclosed mirror-pair structure is applied, a dimensionless symmetry residual derived from pulse-pair amplitude ratios is evaluated, an independent signed eight-slot schedule is verified, and continued emission is permitted or interrupted based on compliance. Narrower fallback method positions are also supported, including exact mirror equality, mirror compliance within a predetermined mismatch tolerance, a preferred mismatch tolerance within plus or minus 0.5 percent, a predetermined residual threshold, a preferred residual threshold of 1/20000, exact preferred threshold values as proof-backed support anchors, one or more direct sensing modalities, optical feedback sensing in a narrower branch, photon-count sensing, optical-intensity sensing, or detector-based pulse verification as preferred direct sensing modalities where implemented, carrier selection relative to the disclosed coherence-quantum criterion, carrier selection satisfying a predetermined harmonic-proximity criterion relative to E_coh, carrier selection satisfying a predetermined energy-band criterion relative to E_coh, visible-carrier selection within the disclosed water-transparency window, visible solid-state emitter implementations selected under the disclosed carrier criteria, micro-LED-based visible emitter implementations as a narrower hardware-class fallback, GaN-based micro-LED implementations as a further narrower hardware-class fallback, a preferred carrier around 450+/−20 nm and within the disclosed preferred tolerance band of the 31st multiple of E_coh, preferred tissue or application classes including dermal tissue, wound-associated tissue, and joint-associated tissue, one or more cycles, a plurality of cycles, and a preferred treatment session of about 200 cycles. The disclosed architecture likewise supports independent system claims centered on an emission source arrangement configured to generate the exact eight-sub-pulse cycle, a controller configured to impose the mirror structure, a verifier configured to compute the dimensionless symmetry residual, a signed-schedule auditor, and a safety control configured to inhibit, pause, temporarily shut down, latched shut down, terminate, or roll back emission on non-compliance. The principal supported system hardware posture remains individually addressable multi-emitter or grouped-emitter architecture, with any single-source branch remaining contingent, secondary, and support-dependent rather than co-equal. Device and product claims are likewise supported by the disclosed hardware and packaging subclasses, including patch-type devices, handheld devices, stationary devices, kits, and software-controlled treatment hardware, while remaining subordinate to the core architecture and not redefining the invention through packaging alone. Computer-readable-medium and firmware claim support is also present in the disclosed control logic. Firmware instructions stored in non-transitory media can cause one or more processors, controllers, verifiers, or associated logic elements to generate eight-sub-burst sequences with the disclosed mirror symmetry, compute the dimensionless J-audit residual, trigger interruption or rollback when compliance fails, verify the signed schedule, apply photon or temperature caps, hash or store telemetry, manage session progression, and enforce calibration-state control. Such claim support is available whether the relevant functions are implemented in hardware logic, firmware, or a combination thereof. The specification therefore preserves support for verifier implementation in hardware logic, firmware, or a combination thereof before narrowing to specific FPGA-plus-microcontroller embodiments.

The emission-source support preserved by the present disclosure includes individually addressable multi-emitter arrays, grouped-emitter arrays configured to generate the exact eight-sub-pulse cycle, visible solid-state emitter implementations selected under the disclosed carrier criteria, micro-LED-based visible emitter implementations as a narrower hardware-class fallback, GaN-based micro-LED implementations as a further narrower hardware-class fallback, and the preferred 450+/−20 nm GaN micro-LED embodiment as a narrower preferred implementation. Individually addressable multi-emitter arrays and grouped-emitter arrays remain the principal supported implementation classes for the disclosed architecture because they directly preserve the exact-cycle and compliance criteria. Any broader controlled sub-pulse generation arrangement is preserved only to the extent it remains tied to those exact-cycle and compliance criteria.

The formal verification material represented by proof architecture 900 also supports claim families directed to protocol correctness, threshold support, carrier-selection support, timing support, and assembled viability certification, while remaining clearly distinct from the empirical pilot data represented by pilot-data summary 1100. The exact preferred threshold values serve as exact, proof-backed support anchors for the preferred threshold regime. The preferred carrier package serves as a proof-backed, non-arbitrary, and architecture-defining preferred implementation package. These proof-backed items are therefore available as claim-support anchors and are not treated as merely illustrative background.

Downstream use-claim support is also present in the disclosure, but remains subordinate and carefully categorized so that downstream indications do not swallow the identity of the invention and are not treated as co-equal with the core method and system claim families. FIG. 12 and FIG. 13 provide preferred downstream embodiment support for wound-associated and joint-associated applications. FIG. 14 and FIG. 15 provide optional downstream embodiment support for stationary and superficial-vascular packaging branches, respectively. FIG. 14 and FIG. 15 remain optional downstream embodiment figures and optional downstream product-claim support branches, and their optionality is preserved consistently so that they may be reduced or removed in a leaner filing posture without destabilizing the core drawing set, the core disclosure, or the core claim architecture.

Certain strategic language should remain strong where support exists because it is tied closely to the load-bearing structure of the invention. Such language includes exactly eight sub-pulses, pairwise mirror symmetry, I_n=I_9-n, phi_n=phi_9-n+pi, dimensionless J-cost symmetry residual, R_sym is dimensionless and does not have units of joules, independent signed eight-slot ledger schedule, equal-and-opposite, ledger-neutral, unique cost functional, exponent 5 is forced, machine-verified, proved, non-thermal, power-capped, and rollback. These terms correspond directly to the identity and distinction of the disclosed architecture and should not be softened into generic pulsed-light phrasing where support exists.

By contrast, certain material should remain subordinate to the core invention. Such subordinate material can include the 450 nm exact carrier value treated as a universal requirement, the 125 Hz exact master frequency treated as a universal requirement, 200 cycles treated as a universal requirement, the 8×8 array size treated as a universal requirement, silver-gel hydrogel composition treated as a universal requirement, specific vendor or part-number references, BLE, smartphone-app, cloud-audit, EHR-integration, and dashboard features, bandage, probe, tabletop, and vascular-patch packaging treated as core identity, particular clinical indications, especially systemic inflammation and collagen remodeling, and commercial or market framing. These items remain useful as dependent-claim support, implementation detail, optional adjunct features, or continuation-worthy targets, but they do not replace the exact audited eight-sub-pulse architecture as the center of the invention.

Likewise, certain items may require more support before being treated as central. Such items can include protein-folding mechanism beyond architecture-level rationale, chaperone-cycle alignment as a demonstrated causal mechanism, I kappa B stabilization as a demonstrated causal mechanism, nuclear-translocation gating as a demonstrated causal mechanism, systemic anti-inflammatory vascular-patch claims for sepsis or SIRS, collagen-remodeling or procollagen-folding centrality, any claim family directed to nucleic acids, vectors, host cells, formulations, or related biotechnology subject matter, and any attempt to import broader Recognition Science scaffold material not specifically tied to this photonic-therapy architecture and its named formal support results. These items can remain available as rationale, prophetic support, or continuation-worthy targets, but they should not be elevated above the disclosure actually provided for the exact audited architecture.

Accordingly, the present disclosure provides a filing-ready non-provisional support structure in which the exact audited eight-sub-pulse architecture is identified as the core inventive center, the figure set progresses from core architecture to implementation and then to downstream embodiments, the control logic preserves distinct compliance states and fault outcomes, the preferred threshold, timing, carrier, and dose regimes are presented as exact, proof-backed, and non-arbitrary preferred implementation packages, the embodiment hierarchy is kept subordinate to the core architecture, prophetic and demonstrated material are segregated, and the resulting disclosure supports broad independent method and system claims together with disciplined fallback positions. The invention is therefore best understood as an exact, audited, cost-balanced, ledger-neutral eight-sub-pulse photonic therapy architecture and the hardware, firmware, verification logic, and treatment methods that enforce that architecture in real time.

What is claimed is:

1. A method of photonic therapy for non-thermal stimulation of biological tissue, the method comprising: generating a treatment cycle comprising exactly eight optical sub-pulses; arranging the eight optical sub-pulses into mirror pairs corresponding to positions (1,8), (2,7), (3,6), and (4,5); controlling the eight optical sub-pulses such that intensities of the mirror pairs satisfy I_n=I_9-n and phases of the mirror pairs satisfy phi_n=phi_9-n+pi; determining a dimensionless symmetry residual R_sym from pulse-pair amplitude ratios of the mirror pairs using a cost functional J(x)=½(x+1/x)−1; verifying an independent signed eight-slot ledger schedule satisfying equal-and-opposite mirror relations across the treatment cycle; and permitting continued optical emission only while (i) the dimensionless symmetry residual satisfies R_sym<=R_tol, where R_tol is a predetermined dimensionless threshold, and (ii) the signed eight-slot ledger schedule satisfies a neutrality condition in which a sum from k=0 to 7 of delta_k equals zero.

2. The method of claim 1, wherein determining the dimensionless symmetry residual comprises determining R_sym=J(I_1/I_8)+J(I_2/I_7)+J(I_3/I_6)+J(I_4/I_5).

3. The method of claim 1, wherein the signed eight-slot ledger schedule satisfies delta_0=−delta_4, delta_1=−delta_5, delta_2=−delta_6, and delta_3=−delta_7.

4. The method of claim 1, wherein the predetermined dimensionless threshold R_tol is selected such that a four-pair aggregate residual corresponding to plus or minus 0.5 percent mirror-pair mismatch remains below R_tol, and wherein the four-pair aggregate residual is 1/20100 and R_tol is 1/20000.

5. The method of claim 1, wherein R_tol is 1/20000 and continued optical emission is interrupted when R_sym>R_tol or when the signed eight-slot ledger schedule fails the neutrality condition.

6. The method of claim 1, wherein the pulse-pair amplitude ratios are determined from measured values for acceptance and fault handling.

7. The method of claim 6, wherein the measured values comprise one or more of photon-count sensing values, optical-intensity sensing values, or detector-based pulse verification values.

8. The method of claim 1, wherein the treatment cycle is delivered using a master timing reference of about 125 Hz, wherein the eight optical sub-pulses are delivered within each treatment cycle at a sub-burst rate of about 1000 Hz, and wherein a treatment session comprises about 200 treatment cycles with an active emission interval of about 1.6 seconds.

9. The method of claim 1, wherein mirror-pair compliance is evaluated against a predetermined mismatch tolerance.

10. The method of claim 9, wherein the predetermined mismatch tolerance is within plus or minus 0.5 percent.

11. The method of claim 1, wherein the treatment cycle is generated using a GaN micro-LED emitter operating at about 450+/−20 nm, and wherein photon energy of the emitter is within a predetermined tolerance band of a 31st multiple of a coherence-quantum criterion E_coh=phi\hat{ }−5.

12. A photonic therapy system comprising: an emission source arrangement configured to generate a treatment cycle comprising exactly eight optical sub-pulses; a controller configured to impose mirror-pair organization across the treatment cycle corresponding to positions (1,8), (2,7), (3,6), and (4,5), and to control the eight optical sub-pulses such that intensities of the mirror pairs satisfy I_n=I_9-n and phases of the mirror pairs satisfy phi_n=phi_9-n+pi; a verifier configured to compute a dimensionless symmetry residual from pulse-pair amplitude ratios using a cost functional J(x)=½(x+1/x)−1, wherein the pulse-pair amplitude ratios are determined from measured pulse values when optical feedback from the photonic therapy system provides direct measurement; a signed-schedule auditor configured to verify an independent signed eight-slot ledger schedule satisfying equal-and-opposite mirror relations across the treatment cycle; and a safety control configured to permit continued optical emission only while (i) the dimensionless symmetry residual satisfies R_sym<=R_tol, where R_tol is a predetermined dimensionless threshold, and (ii) the signed eight-slot ledger schedule satisfies a neutrality condition in which a sum from k=0 to 7 of delta_k equals zero, and to interrupt emission when R_sym>R_tol or when the signed eight-slot ledger schedule fails the neutrality condition.

13. The system of claim 12, wherein the emission source arrangement comprises an individually addressable multi-emitter array controlled through DAC-based drive control.

14. The system of claim 12, wherein the verifier is configured to compare commanded pulse values with measured pulse values and store commanded-versus-measured deviation information for diagnostic, calibration, or compliance analysis.

15. A non-transitory computer-readable medium storing instructions that, when executed by one or more processors of a photonic therapy apparatus, cause the apparatus to generate a treatment cycle comprising exactly eight optical sub-pulses, arrange the eight optical sub-pulses into mirror pairs corresponding to positions (1,8), (2,7), (3,6), and (4,5), compute a dimensionless symmetry residual R_sym from pulse-pair amplitude ratios of the mirror pairs using a cost functional J(x)=½(x+1/x)−1, verify an independent signed eight-slot ledger schedule satisfying a neutrality condition in which a sum from k=0 to 7 of delta_k equals zero, use measured pulse values for acceptance and fault handling when optical feedback from the photonic therapy apparatus provides direct measurement, inhibit or interrupt emission when R_sym>R_tol, where R_tol is a predetermined dimensionless threshold, or when the signed eight-slot ledger schedule fails the neutrality condition, store residual-threshold margin information, and generate a hashed audit record, a signed audit record, or a hashed and signed audit record.

16. The system of claim 12, wherein the emission source arrangement comprises a GaN micro-LED implementation operating at about 450+/−20 nm.

17. The system of claim 12, wherein the photonic therapy system is configured as a wearable or adhesive wound-treatment device comprising a bandage body, an emitter region positioned to deliver the treatment cycle toward wound-associated tissue, and a sterile or single-use hydrogel interface positioned between the emitter region and the wound-associated tissue.

18. The system of claim 12, further comprising an optical feedback subsystem configured to provide one or more of photon-count sensing, optical-intensity sensing, or detector-based pulse verification, wherein the verifier is configured to use measured pulse values from the optical feedback subsystem for acceptance and fault handling when direct measurement is available.

19. The system of claim 12, further comprising rollback circuitry configured to interrupt emission within about 200 ns after detection of one or more fault conditions comprising R_sym>R_tol, failure of the signed eight-slot ledger schedule to satisfy the neutrality condition, a photon-budget exceedance relative to one or more photon caps comprising at most 1.5×10\hat{ }15 photons per sub-pulse, at most 1.2×10\hat{ }16 photons per cycle, or at most 2.4×10\hat{ }18 photons per treatment session, a temperature-limit exceedance, a calibration-invalid state, or a watchdog fault.

20. The system of claim 12, further comprising a coupling interface comprising a hydrogel interface configured to provide optical coupling and thermal buffering at a tissue interface such that a temperature rise at the tissue interface remains less than about 0.3 C during a treatment session.
